(19) United States
(12) Patent Application Publication
(10) Pub. No.: US 2006/0141010 A1
(43) Pub. Date: Jun. 29, 2006

(54) Coated preparations that contain at least one hydroformate

(75) Inventors: Jorn Karl, Ludwigshafen (DE); Gerd Diebold, Reutlingen (DE); Andreas Habich, Speyer (DE); Holger Barthel, Schwetzingen (DE)

Correspondence Address: CONNOLLY BOVE LODGE & HUTZ, LLP, P O BOX 2207, WILMINGTON, DE, 19899, US

(73) Assignee: Basf Aktiengesellschaft, Ludwigshafen (DE)

(21) Appl. No.: 10/561,704

(22) PCT Filed: Jun. 11, 2004
(86) PCT No.: PCT/EP04/06296

§ 371 Date: Dec. 22, 2005

(30) Foreign Application Priority Data
Jun. 24, 2003 (DE) 10328519.9
Mar. 9, 2004 (DE) 102004011832.9

(51) Int. Cl. A01N 59/04; A23K 1/165; A61K 31/19

(52) U.S. Cl. 424/442; 514/557; 504/101

(57) ABSTRACT

The present invention relates to coated or liquid preparations comprising at least one hydroformate of the general formula (I)
M₃[HCOO]₃.HCOOH (I)
where M=Na, K, Cs, NH₄, and the use thereof.

The present invention relates to coated or liquid preparations comprising at least one hydroformate and also to the use of these preparations.

Acid formates and methods for preparing these have long been known. Thus, Gmelins Handbuch der anorganischen Chemie [Gmelin's handbook of inorganic chemistry], 8th Edition, number 21, pages 816 to 819, Verlag Chemie GmbH, Berlin 1928, and number 22, pages 919 to 921, Verlag Chemie GmbH, Berlin 1937, describes the synthesis of sodium diformate, and of potassium diformate, by dissolving sodium formate, and potassium formate, in formic acid. The crystalline diformates are available by lowering the temperature or by evaporating off excess formic acid.

DE 424017 teaches preparing acid sodium formates having differing acid content by introducing sodium formate into aqueous formic acid in an appropriate molar ratio. By cooling the solution the corresponding crystals can be obtained.

According to J. Kendall et al., Journal of the American Chemical Society, Vol. 43, 1921, pages 1470 to 1481, acid potassium formates may be obtained by dissolving potassium carbonate in 90% strength formic acid, with formation of carbon dioxide. The corresponding solids can be obtained by crystallization.

GB 1,505,388 discloses preparing acid carboxylate solutions by mixing the carboxylic acid with a basic compound of the desired cation in aqueous solution. Thus, for example, in the preparation of acid ammonium carboxylate solutions, ammonia water is used as basic compound.

U.S. Pat. No. 4,261,755 describes the preparation of acid formates by reacting an excess of formic acid with the hydroxide, carbonate or bicarbonate of the corresponding cation.

WO 96/35657 teaches the preparation of products which contain disalts of formic acid by mixing potassium, sodium, cesium or ammonium formate, potassium, sodium or cesium hydroxide, potassium, sodium or cesium carbonate or bicarbonate or ammonia with possibly aqueous formic acid, subsequently cooling the reaction mixture, filtering the resultant suspension and drying the resultant filtercake and recirculating the filtrate.

The unpublished German applications DE 101 547 15.3 and DE 102 107 30.0 describe processes for preparing formates.

Acid formates have an antimicrobial action and are used, for example, for preserving and acidifying plant and animal materials, for instance grasses, agricultural products or meat, for treating biowastes or as additive for animal nutrition.

WO 96/35337 A1 describes animal feedstuffs and animal feed additives which comprise diformates, in particular potassium diformate.

WO 97/05783 A1 (EP 845 947 A1) describes a process for cooling and preserving fish in which a cooling medium containing formic acid and/or mono/di or tetrasalts of formic acid is used. In one embodiment, a C1 to C4 monocarboxylic acid is added to the cooling medium.

WO 98/19560 (EP 957 690 A1) describes a process for preparing a fish food in which ammonium, sodium or potassium diformate and formic acid are added to a fish products before the other fish food constituents are added and they are processed into fish food.

WO 98/20911 A1 (EP 961 620 B1) describes a process for treating moist organic waste in which an aqueous preparation of the monosalts and disalts of formate, acetate or propionate is used.

WO 01/19207 A1 describes a liquid preservative/acidifier for grass and agricultural products, fish and fish products and meat products which comprises at least 50% by weight of formic acid and formates, ammonium tetraformate and 2-6% by weight of potassium or 2-10% by weight of sodium in the form of their hydroxides or formates.

The diformates available according to EP 0 824 511 B1 can be dried in a step following the preparation process. This produces products which are powders and generally have less than 5% by weight of water. However, the diformates which are thus obtainable are unsuitable for formulation in complex substrates.

Commercially available preparations comprising diformate, as are obtainable, for example, under the name FORMI™ for animal nutrition, cannot in general be incorporated into premixes, since lump formation and or caking of the premix occurs which makes further processing of the premix to feeds impossible.

In the production of feeds, vitamins, minerals, trace elements, organic acid and optionally enzymes are prepared in the form of what are termed premixes or base mixes and then mixed with the remaining constituents of the feed. If the diformates which are known from the prior art (for example according to EP 0 824 511 B1) are mixed into a premix, lump formation and caking occur and also breakdown of constituents of the premix.

It is an object of the present invention to provide preparations which make it possible to incorporate hydroformates in complex substrates, for example in animal feeds or premixes for animal feeds, without lump formation or aggregation of the substrate occurring. It is of particular interest here that the remaining constituents of the complex substrate are not affected by the admixture. Particular importance among the complex substrates is attached to what are termed base mixes, as are customarily used for feed production.

It is also an object of the present invention to prepare formulations of hydroformates which, when used in feeds, prevent premature release of formic acid salts in the stomach.

We have found that this object is achieved by the inventive coated preparations.

The invention therefore relates to coated preparations comprising at least one hydroformate of the general formula I
M₃[HCOO]₃.HCOOH (I)
where M=Na, K, Cs, NH₄

The term “coated preparations” here comprises all preparations where the surface of the particles of the preparation is at least 50%, in particular at least 70%, very particularly preferably at least 80%, in particular at least 90%, covered. The terms “coated”, “encapsulated”, “powdered” and “encased” are used synonymously in the meaning of the present application. Correspondingly, the terms coating material, powder-coating material, encasing material and coating agent are used synonymously.

Coated preparations are, in particular, those preparations which, in contact with calcium carbonate, remain substantially chemically unchanged compared with uncoated preparations. A parameter of a chemical change of the hydroformates is the release of CO₂.

The invention comprises preparations comprising at least one hydroformate of the general formula (i)
M₃[HCOO]₃.HCOOH (I)
where M=Na, K, Cs, NH₄, and
   at least one coating material.
   Hydroformates

The compounds of the formula (I) are termed either hydroformates or tetraformates. A further customary name is trisodium/potassium/cesium/ammonium hydrogenformate.

The synthesis of Na₃[HCOO]₃.HCOOH is described in Gmelins Handbuch der organischen Chemie, 8th Edition, sodium, p. 21. The hydroformates to be used according to the invention are, for example, available by the process described in EP 0 824 511 B1, or by the process described in the still unpublished German patent applications DE 101 547 15.3 and DE 102 107 30.0.

Those which are to be used according to the invention are Na₃[HCOO]₃.HCOOH (trisodium hydrogenformate), K₃[HCOO]₃.HCOOH (tripotassium hydrogenformate), Cs₃[HCOO]₃.HCOOH (tricesium hydrogenformate), NH₃[HCOO]₃.HCOOH (triammonium hydrogenformate).

A particularly preferred hydroformate is Na₃[HCOO]₃.HCOOH (trisodium hydrogenformate).

In a further embodiment, said hydroformates can be used in mixtures with one another.

The term “preparation comprising hydroformates” is used hereinafter and comprises not only preparations comprising at least one hydroformate, but also preparations comprising mixtures of said hydroformates.

The hydroformates are used in the coated preparations customarily in amounts of from 0.01 to 30% by weight, in particular from 0.1 to 20% by weight, preferably from 1 to 5% by weight, in particular from 0.3 to 5% by weight, based on the total preparation.

Liquid Preparations

The present invention further relates to liquid preparations comprising hydroformates. In a preferred embodiment, an aqueous solution comprising hydroformates is used. The liquid preparations comprise from 0.01 to 60% by weight, in particular from 0.01 to 50% by weight, in particular from 1 to 40% by weight, preferably from 5 to 30% by weight, of hydroformates. A person skilled in the art will choose the content of hydroformate in the liquid preparation as a function of the solubility of the individual hydroformates or of the mixtures of hydroformates.

The present invention relates in particular to liquid preparations comprising
from 0.01 to 60% by weight of at least one hydroformate of the general formula (I)
   M₃[HCOO]₃.HCOOH (I)
   where M=Na, K, Cs, NH₄, and
from 99.99 to 40% by weight of water
   with the proviso that the percentages by weight total 100%.

The liquid preparations can comprise, in addition to the solvent and the hydroformate, other customary aids. The content of aids is determined according to the solubility of the respective aids. The total of the aids is customarily from 0 to 50% by weight, preferably from 0 to 20% by weight, in particular from 1 to 10% by weight, based on the weight of the liquid preparation (liquid and hydroformate).

Coating Materials

Coating materials which can be used are all materials which are able to cover the surface of preparations comprising at least one hydroformate by at least 50%, in particular at least 70%, very particularly preferably at least 80%, in particular at least 90%.

Coating materials which can be used are all materials which are able to coat preparations comprising at least one hydroformate in such a manner that these remain chemically unchanged in contact with calcium carbonate. A parameter of a chemical change of the hydroformates is the release of CO₂. This is determined by the following method:

20 g each of feed lime and sample are added, mixed, to a 100 ml conical flask and this is connected via an attachment head with tubing to a water-filled measuring cylinder which is suspended upside down. The conical flask is shaken at 500 rpm on a shaking plate and the resultant CO₂ is collected in the measuring cylinder. The measurement is carried out at room temperature (20° C.). The release of CO₂ is measured over a period of 200, 600 and 800 minutes. Suitable coating materials are all compounds which result in coated preparations comprising at least one hydroformate in which release of CO₂ after 200 minutes is less than 100 ml, in particular less than 50 ml of CO₂, in particular less than 25 ml of CO₂.

A person skilled in the art will choose the coating method as a function of the coating material used.

A person skilled in the art will choose the amount of coating material as a function of the coating material selected in such a manner that the coating material covers at least 50%, in particular at least 70%, very particularly preferably at least 80%, in particular at least 90%, of the surface of the preparation to be coated. Customary amounts of coating material used here are, for example, from 2 to 50% by weight, based on the preparations, in particular from 3 to 35%, preferably from 5 to 20% by weight.

In a preferred embodiment, the inventively coated preparations are provided with a coating material which comprises at least one compound which is selected from the group consisting of
a) polyalkylene glycols, in particular polyethylene glycols, having a number-average molecular weight of from about 400 to 15 000, for example from 400 to 10 000;
b) poly(alkylene oxide) polymers or copolymers having a number-average molecular weight of from about 4000 to 20 000, in particular block copolymers of polyoxyethylene and polyoxypropylene;
c) substituted polystyrenes, maleic acid derivatives and also styrene-maleic acid copolymers;
d) polyvinylpyrrolidones having a number-average molecular weight of from about 7000 to 1 000 000;
e) vinylpyrrolidone/vinyl acetate copolymers having a number-average molecular weight of from about 30 000 to 100 000;
f) poly(vinyl alcohol) having a number-average molecular weight of from about 10 000 to 200 000, poly(vinyl phthalate)s;
g) hyroxypropylmethylcellulose having a number-average molecular weight of from about 6000 to 80 000;
h) alkyl (meth)acrylate polymers and copolymers having a number-average molecular weight of from about 100 000 to 1 000 000, in particular ethyl acrylate/methyl methacrylate copolymers and methacrylate/ethyl acrylate copolymers;
i) poly(vinyl acetate) having a number-average molecular weight of from about 250 000 to 700 000, optionally stabilized with polyvinylpyrrolidone;
j) polyalkylenes, in particular polyethylenes;
k) phenoxyacetic acid-formaldehyde resin;
l) cellulose derivatives, such as ethylcellulose, ethylmethylcellulose, methylcellulose, hydroxypropylcellulose, hydroxypropylmethylcellulose, carboxymethylcellulose, cellulose acetate phthalate;
m) animal, vegetable or synthetic fats;
n) animal, plant or synthetic waxes or chemically modified animal, plant waxes such as beeswax, candelilla wax, carnauba wax, montan ester wax and rice germ oil wax, spermaceti, lanolin, jojoba wax, sasol wax, Japan wax or Japan wax substitute;
o) animal and plant proteins, for example gelatin, gelatin derivatives, gelatin substitutes, casein, whey, keratin, soybean protein; zein and wheat protein;
p) mono- and disaccharides, oligosaccharides, polysaccharides, for example starches, modified starches and also pectins, alginates, chitosan, carrageenans;
q) vegetable oils, for example sunflower oil, thistle oil, cottonseed oil, soybean oil, corn germ oil, olive oil, rape(seed) oil, linseed oil, coconut oil, palm kernel oil, and palm oil;
r) synthetic or semisynthetic oils, for example medium-chain triglycerides or mineral oils;
s) animal oils, for example herring oil, sardine oil and whale oil;
t) hardened (hydrogenated or partially hydrogenated) oils/fats, for example of the abovementioned, in particular hydrogenated palm oil, hydrogenated cottonseed oil, hydrogenated soybean oil;
u) lacquer coatings, for example terpenes, in particular shellac, Tolu balsam, Peru balsam, sandarac and silicone resins;
v) fatty acids, not only saturated but also monounsaturated and polyunsaturated C₆- to C₂₄-carboxylic acids;
w) silicic acids;
x) benzoic acid and/or salts of benzoic acid and/or esters of benzoic acid and/or derivatives of benzoic acid and/or salts of benzoic acid derivatives and/or esters of benzoic acid derivatives.

Said coating materials can also be used in mixtures with one another.

Examples of suitable poly(alkylene glycol)s a) are: poly(propylene glycol)s and in particular poly(ethylene glycol)s of differing molar mass, for example PEG 4000 or PEG 6000, obtainable from BASF Aktiengesellschaft under the trade names Lutrol E 4000 and Lutrol E 6000.

Examples of the above polymers b) are: poly(ethylene oxide)s and poly(propylene oxide)s, ethylene oxide/propylene oxide mixed polymers and block copolymers, made up of poly(ethylene oxide) and poly(propylene oxide) blocks, for example polymers which are obtainable from BASF Aktiengesellschaft under the trade name Lutrol F68 and Lutrol F127.

Of the polymers a) and b), preferably highly concentrated solutions of up to about 50% by weight, for example from about 30 to 50% by weight, based on the total weight of the solution, can advantageously be used.

Examples of above polymers d) are: polyvinylpyrrolidones, as are distributed, for example, by BASF Aktiengesellschaft under the trade name Kollidon or Luviskol. Of these polymers, highly concentrated solutions having a solids content of from about 30 to 40% by weight, based on the total weight of the solution, can advantageously be used.

An example of abovementioned polymers e) is: a vinylpyrrolidone/vinyl acetate copolymer which is distributed by BASF AG under the trade name Kollidon VA64 or Kollicoat SR. Of these copolymers, highly concentrated solutions of from about 30 to 40% by weight, based on the total weight of the solution, can particularly advantageously be used.

Examples of above polymers f) are: products, as are distributed, for example, by Hoechst under the trade name Mowiol. Of these polymers, solutions having a solids content in the range of from about 8 to 20% by weight can advantageously be used.

Examples of suitable polymers g) are: hydroxypropylmethylcelluloses, as are distributed, for example, by Shin Etsu under the trade name Pharmacoat.

Examples of abovementioned polymers h) are: alkyl (meth)acrylate polymers and copolymers, the alkyl group of which has from 1 to 4 carbon atoms. Specific examples of suitable copolymers are: ethyl acrylate/methyl methacrylate copolymers, which are distributed, for example, under the trade names Kollicoat EMM 30D by BASF AG or under the trade name Eudragit NE 30 D by Rbhm; and also methacrylate/ethyl acrylate copolymers as are distributed, for example, under the trade name Kollicoat MAE 3ODP by BASF Aktiengesellschaft or under the trade name Eudragit 30/55 by Rbhm. Copolymers of this type can be processed inventively, for example, as from 10 to 40% strength by weight dispersions.

Examples of above polymers i) are: poly(vinyl acetate) dispersions which are stabilized with polyvinylpyrrolidone and are distributed, for example, under the trade name Kollicoat SR 30D by BASF Aktiengesellschaft (solids content of the dispersion of from about 20 to 30% by weight.

Fats, for example those of animal, vegetable or synthetic origin; examples of animal fats m) are fats of pigs, cattle and geese, beef tallow, for example, is suitable. A suitable beef tallow is available under the trade name Edenor NHIT-G (CAS No. 67701-27-3) from Cognis.

Other coating materials are gelatin, for example from cattle, from pigs, from fish.

Other coating materials are waxes, for example plant waxes, for example candelilla wax, carnauba wax, rice germ oil wax, Japan wax or Japan wax substitute (available under the trade name Japan wax substitute 2909, Kahl Wachsraffinerie) etc.; synthetic waxes such as cetyl palmitate (obtainable under the trade name Cutina CP, CAS 95912-87-1 from Cognis), animal waxes, for example lanolin, beeswax, shellac wax, spermaceti and also chemically modified waxes, such as jojoba wax, sasol wax, montan ester wax.

In principle, other coatings from solution are also conceivable: for example sugar coating.

Likewise, plant oils q), for example sunflower oil, thistle oil, cottonseed oil, soybean oil, corn germ oil and olive oil, rape oil, linseed oil, coconut, (oil) palm kernel oil and (oil) palm oil; can be used. Suitable palm oils are obtainable, for example, under the trade name Vegeol PR 265 from Aarhus Oliefabrik. Suitable rape(seed) oils are obtainable under the trade name Vegeol PR 267 from Aarhus Oliefabrik. Palm kernel oil is obtainable under the trade name Tefacid Palmic 90 (CAS No. 57-10-3) from Karlshamns.

Likewise, semisynthetic oils r) can be used, for example medium-chain triglycerides or mineral oils and/or animal oils s), for example herring oils, sardine oils and whale oils.

In a preferred embodiment, as coating material, use is made of hydrogenated vegetable oils t) including triglycerides, for example hydrogenated cottonseed oil, corn oil, peanut oil, soybean oil, palm oil, palm kernel oil, babassu oil, sunflower oil, and safflower oil. Preferred hydrogenated vegetable oils comprise hydrogenated plam oil, cottonseed oil and soybean oil. The most preferred hydrogenated vegetable oil is hydrogenated soybean oil. Other fats and waxes originating from plants and animals are likewise suitable.

The preferred hydrogenated vegetable oils can be present in various polymorphous forms; these are the α, β and β′ forms. In a particularly preferred embodiment of the present invention, hydrogenated vegetable oils are used which are predominantly in the β and β′ form, in particular those which are predominantly in the β form. The term “predominantly” means that at least 25%, in particular at least 50%, preferably at least 75%, of the crystals are in the preferred polymorphous form.

Particular preference is given to the use of hydrogenated soybean oil having a portion greater than 50%, in particular greater than 75%, preferably greater than 90%, β and/or β′ form.

In a preferred embodiment, the inventively coated preparations are provided with a coating material which comprises at least one compound which is selected from the group consisting of benzoic acid and/or salts of benzoic acid and/or esters of benzoic acid and/or derivatives of benzoic acid and/or salts of benzoic acid derivatives and/or esters of benzoic acid derivatives.

Salts of benzoic acid or of benzoic acid derivatives are alkali metal salts and/or alkaline earth metal salts of benzoic acid and also ammonium benzoate. Alkali metal salts are: lithium, sodium, potassium and cesium benzoates. Particular preference is given to sodium and/or potassium benzoates. Alkaline earth metal salts are calcium, strontium and magnesium benzoates, particular preference is given to calcium and magnesium benzoates.

Esters of benzoic acid or of benzoic acid derivatives are the esters of benzoic acid or of benzoic acid derivatives with alcohols. Suitable alcohols are not only monofunctional but also bifunctional and polyfunctional (more than 2 hydroxyl groups). Suitable alcohols are not only unbranched but also branched alcohols. Particularly suitable alcohols are those having from 1 to 10 carbon atoms, in particular having from 1 to 6 carbon atoms. Examples are: methanol, ethanol, n-propanol, isopropanol, n-butyl alcohol, isobutyl alcohol. Preference is given to methanol, ethanol, n-propanol and isopropanol. Suitable esters of benzoic acid or of benzoic acid derivatives are, in addition, esters with alcohols having more than one hydroxyl group, for example glycols, examples being 1,2-propanediol or triols, for example glycerol.

Preference is given to methyl benzoate, ethyl benzoate, n-propyl benzoate and isopropyl benzoate and also to ethyl p-hydroxybenzoate, sodium methyl p-hydroxy-benzoate, propyl p-hydroxybenzoate, sodium propyl p-hydroxybenzoate, methyl p-hydroxybenzoate and sodium methyl p-hydroxybenzoate.

Derivatives of benzoic acid are compounds which bear one, two, three, four or five substituents on the aromatic ring.

The substituents of the inventive carboxylic acids are, for example, C₁-C₈-alkyl, C₂-C₈-alkenyl, aryl, aralkyl and aralkenyl, hydroxymethyl, C₂-C₈-hydroxyalkyl, C₂-C₈-hydroxy-alkenyl, aminomethyl, C₂-C₈-aminoalkyl, cyano, formyl, oxo, thioxo, hydroxy, mercapto, amino, carboxy or imino groups. Preferred substituents are C₁-C₈-alkyl, hydroxymethyl, hydroxyl, amino and carboxyl groups.

Preferred benzoic acid derivatives are mono-, di- and trihydroxy substituted benzoic acids.

Examples are m-hydroxybenzoic acid, o-hydroxybenzoic acid, and p-hydroxybenzoic acid.

Examples are 2,4-dihydroxybenzoic acid, 2,5-dihydroxybenzoic acid, 2,5-dihydroxybenzoic acid, 2,6-dihydroxybenzoic acid, 3,5-dihydroxybenzoic acid, 3,6-dihydroxybenzoic acid, 2,4,6-trihydroxybenzoic acid.

Particular preference is given to monohydroxy-substituted benzoic acids, in particular p-hydroxybenzoic acid.

In a further embodiment, said benzoates can be used in a mixture with one another.

Preferred coating materials comprise hydrogenated vegetable oils including triglycerides, for example hydrogenated cottonseed oil, corn oil, peanut oil, soybean oil, palm oil, palm kernel oil, babassu oil, sunflower oil and safflower oil. Preferred hydrogenated vegetable oils comprise hydrogenated palm oil, cottonseed oil and soybean oil. The most preferred hydrogenated vegetable oil is hydrogenated soybean oil. Other fats and waxes originating from plants and animals are likewise suitable.

The table below contains particularly suitable coating materials

| Name | Composition | Melting range | CAS No./INCI |
|---|---|---|---|
| Cutina CP from | Synthetic cetyl palmitate | 46-51° C. | 95912-87-1 |
| Cognis | | | Cetyl Palmitate |
| Edenor NHTI-G from | Triglyceride | 56-60° C. | 67701-27-3* |
| Cognis | | | |
| Edenor NHTI-V from | Triglyceride | 57-60° C. | 67701-27-3* |
| Cognis | | | EINECS 266-945-8 |
| Japan wax substitute | Complex mixture of wax esters, | 49-55° C. | rhus succedanea |
| Kahl - Wachsraffinerie | principally palmitic acid glycerol ester | | |
| Vegeol PR-267 from | 25% Colzawar 46, 75% Rucawar FH | 70° C. | |
| AARHUS OLIE | | | |
| Vegeol PR-272 from | 80% Shoguwar FH, 20% Shoguwar 41 | 67° C. | |
| AARHUS OLIE | | | |
| Vegeol PR-273 from | 85% Shoguwar FH, 15% Shoguwar 41 | 67° C. | |
| AARHUS OLIE | | | |
| Vegeol PR-274 from | 90% Shoguwar FH, 10% Shoguwar 41 | 67° C. | |
| AARHUS OLIE | | | |
| Vegeol PR-275 from | 95% Shoguwar FH, 5% Shoguwar 41 | 67° C. | |
| AARHUS OLIE | | | |
| Vegeol PR-276 from | 80% Soyalu FH, 20% Soyalu 41 | 65° C. | |
| AARHUS OLIE | re-esterified | | |
| Vegeol PR-277 from | 85% Soyalu FH, 15% Soyalu 41 | 65° C. | |
| AARHUS OLIE | re-esterified | | |
| Vegeol PR-278 from | 90% Soyalu FH, 10% Soyalu 41 | 66° C. | |
| AARHUS OLIE | re-esterified | | |
| Vegeol PR-279 from | 95% Soyalu FH, 5% Soyalu 41 | 67° C. | |
| AARHUS OLIE | re-esterified | | |
| Tefacid from | Palm kernel oil Tefacid Palmic 90 | 65° C. | 57-10-3 |
| Karlshamns | | | |
| Soybean fat powder | | 65-70° C. | |
| from Sankyu/Japan | | | |
| Kollicoat EMM 30 D | 2-Propenoic acid, 2-methyl-, methyl | | 9010-88-2 |
| from BASF | ester | | |
| | polymer with ethyl 2-propenoate | | |
| Kollicoat MAE | Ethyl acrylate-methacrylic acid- | | 25212-88-8 |
| from BASF | copolymer | | Acrylates |
| | | | Copolymer |
| Acronal S 600 | Styrene-acrylic acid ester- | | |
| from BASF | copolymer | | |
| Diofan 233 D | Vinylidene chloride-methyl | | |
| from BASF | acrylate-copolymer | | |
| Poligen WE 3 | Ethylene-acrylic acid- | | |
| from BASF | copolymer | | |
| Dispersion 1286 | polyethylene dispersion in water | | |
| from Paramelt | | | |
| Aquasil PE 1286 | | | |

The coating materials can be used not only individually but also in mixtures with one another. In an embodiment of the invention talcum and/or aluminosilicates, for example zeolites, feldspars or feldspathoids are added to the coating materials. Talcum=Mg₃(OH)₂/Si₄O_{10.}, also called soapstone.

For the coating, use can be made of, for example, an as highly concentrated as possible still sprayable liquid, for example an up to 50% strength by weight aqueous or nonaqueous solution or dispersion of one or more of said coating materials. Likewise, pulverulent coating materials can be used.

In a further embodiment, the inventive coated or liquid preparations, in addition to the hydroformates, can comprise other constituents. The choice of the other constituents depends here on the chosen field of use of the preparations thus obtainable. Further constituents in the meaning of the present invention are, for example, the following substances: organic acids, vitamins, carotenoids, trace elements, antioxidants, enzymes, amino acids, minerals, emulsifiers, stabilizers, preservatives, binders, anticaking agents and/or flavor substances.

In a preferred embodiment, the inventive coated or liquid preparations can in addition comprise at least one short-chain carboxylic acid and/or at least one salt of the short-chain carboxylic acid and/or at least one ester of the short-chain carboxylic acid and/or at least one derivative of the short-chain carboxylic acid.

For the purposes of the present invention, short-chain carboxylic acids are carboxylic acids which can be saturated or unsaturated and/or unbranched or branched or cyclic and/or aromatic and/or heterocyclic. For the purposes of the invention, “short-chain” means carboxylic acids which contain up to 12 carbon atoms, in particular up to 10 carbon atoms, in particular up to 8 carbon atoms.

The short-chain carboxylic acids customarily have a molecular weight less than 750. The short-chain carboxylic acids for the purposes of the present invention can have one, two, three or more carboxyl groups. The carboxyl groups can be present wholly or partly as ester, acid anhydride, lactone, amide, imidic acid, lactam, lactim, dicarboximide, carbohydrazide, hydrazone, hydrocam, hydroxime, amidine, amidoxime or nitrile.

Derivatives of the short-chain carboxylic acids are carboxylic acids which are monosubstituted, disubstituted, trisubstituted or multiply substituted along the carbon chain or ring.

Substituents of the inventive carboxylic acids are, for example, C1-C8-alkyl, C2-C8-alkenyl, aryl, aralkyl and aralkenyl, hydroxymethyl, C2-C8-hydroxyalkyl, C2-C8-hydroxyalkenyl, aminomethyl, C2-C8-aminoalkyl, cyano, formyl, oxo, thioxo, hydroxy, mercapto, amino, carboxyl or imino groups. Preferred substituents are C1-C8-alkyl, hydroxymethyl, hydroxy, amino and carboxyl groups.

Examples of inventive short-chain carboxylic acids are formic acid, acetic acid, propionic acid, butyric acid, lactic acid, citric acid, isobutyric acid, valeric acid, isovaleric acid, pivalic acid, oxalic acid, malonic acid, salicylic acid, tartaric acid, succinic acid, glutaric acid, glyceric acid, glyoxilic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, o,m,p-phthalic acid, naphthoic acid, toluylic acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolecarboxylic acid, 1,2,4,6,7-naphthalenepentaacetic acid, malonaldehydic acid, 4-hydroxyphthalamidic acid, 1-pyrazolecarboxylic acid, gallic acid or propanetricarboxylic acid.

Salts of the short-chain carboxylic acids which may be mentioned are alkali metal salts and/or alkaline earth metal salts and also ammonium salts. Alkali metal salts which may be mentioned are: lithium, sodium, potassium and cesium salts. Particular preference is given to sodium and/or potassium salts. Alkaline earth metal salts which may be mentioned are calcium, strontium and magnesium salts, particular preference is given to calcium and magnesium salts.

Esters of the short-chain carboxylic acids which may be mentioned are the esters with alcohols. Suitable alcohols are not only monofunctional but also bifunctional and also polyfunctional (more than 2 hydroxyl groups). Suitable alcohols are not only unbranched but also branched alcohols. Particularly suitable alcohols are those having from 1 to 10 carbon atoms, in particular having from 1 to 6 carbon atoms. Examples are: methanol, ethanol, n-propanol, isopropanol, n-butyl alcohol, isobutyl alcohol. Preference is given to methanol, ethanol, n-propanol and isopropanol. Suitable esters are, in addition, esters with alcohols having more than one hydroxyl group, for example glycols; examples are 1,2-propanediol or triols, for example glycerol.

Preferred esters are methyl, ethyl, n-propyl and isopropyl esters.

Particular preference is given to the use of acids and/or salts and/or esters of formic acid, acetic acid, propionic acid, fumaric acid, salicylic acid, citric acid, lactic acid and/or tartaric acid.

Very particular preference is given to the use of sodium propionate.

Very particular preference is given to the use of short-chain carboxylic acids which are described as coating materials w).

In a further embodiment, said short-chain carboxylic acids, salts and/or esters can be used in mixtures with one another.

Suitable compounds are the compounds mentioned under coating materials x).

In a further embodiment, the inventive coated preparations described, in addition to the hydroformates, can comprise supports. In this embodiment the hydroformates are preferably bound to the support. Suitable supports are “inert” support materials, that is to say materials which do not exhibit any adverse interactions with the components used in the inventive preparation. Of course, the support material must be harmless for the respective uses as aid, for example in animal feeds. Suitable support materials are not only inorganic but also organic supports. Examples of suitable support materials which may be mentioned are: low-molecular-weight inorganic or organic compounds and also higher-molecular-weight organic compounds of natural or synthetic origin. Examples of suitable low-molecular-weight inorganic supports are salts, such as sodium chloride, calcium carbonate, sodium sulfate and magnesium sulfate, kieselguhr or silicic acid or silicic acid derivatives, for example silicon dioxides, silicates or silica gels. Examples of suitable organic supports are, in particular, sugars, for example glucose, fructose, sucrose, and also dextrins and starch products. Examples of higher-molecular-weight organic supports are: starch and cellulose preparations, such as in particular corn starch, corncob flour, ground rice hulls, wheat semolina bran or cereal flours, for example wheat, rye, barley and oat flour or bran or mixtures thereof.

In a further embodiment, the inventive coated preparations, in addition to the hydroformates, can contain additives. “Additives” are substances which act to improve the product properties, such as dusting behavior, flow properties, water absorption capacity and storage stability. Additives and/or mixtures thereof can be on the basis of sugars for example lactose or maltodextrin, on the basis of cereal products or pulse products, for example corncob flour, wheat bran and soybean meal, on a basis of mineral salts, inter alia calcium, magnesium, sodium, potassium salts and also D-pantothenic acid or salts thereof themselves (D-pantothenic acid salt prepared chemically or by fermentation).

The inventive coated preparations can comprise the further constituents, supports and additives in mixtures.

The inventive coated preparations are customarily in solid form, for example powders, agglomerates, adsorbates, granules and/or extrudates. The powders usually have a mean particle size of from 1 μm to 10 000 μm, in particular from 20 μm to 5000 μm.

The mean particle size distribution is determined as follows:

The pulverulent products are studied on an instrument from Malvern Instruments GmbH, Mastersizer S, Serial Number: 32734-08. To describe the width of the particle size distribution, for the powders the values D(v,0.1), D(v,0.5) and D(v,0.9) are determined and also the mean particle size of the distribution D[4,3] is reported.

Preparation Processes

For preparing the inventive coated preparations, all processes are suitable in which preparations are obtained, the surface area of which is at least 50%, in particular at least 70%, very particularly preferably at least 80%, in particular at least 90% covered.

The present invention relates to a process for preparing the inventive coated preparations in which the hydroformates are coated by desublimation of the coating material.

In this process the coating material is sublimed and desublimated, that is to say precipitated, on the preparations to be coated. Such processes are known from the literature as sublimation or desublimation processes. The inventive process makes it possible to apply the coating material homogeneously and in desired layer thicknesses. The process of sublimation and desublimation is described in Ullmann's Encyclopedia of Industrial Chemistry, Sixth Edition, 2000 Electronic Release, chapter 4.1. Suitable evaporators (sublimators) are those as described in Ullmann's work cited above, chapter 5.1, and condensers (desublimators) are as described in chapter 5.2; apparatus designs and circuits are described in FIGS. 5,6,7, 9 and 10 which are expressly incorporated herein by reference. A further possible condenser is the fluidized bed.

In a preferred embodiment, the coating material used in this process is at least one compound from the material class x).

The present invention relates to a process for preparing coated preparations comprising hydroformates, in which the hydroformates, optionally together with further constituents and/or additives, are charged in a suitable apparatus and coated with a coating material, optionally with addition of further constituents.

Examples of suitable apparatuses are: mixer, fluidized bed, dragee drums and drum coaters.

The hydroformates, advantageously in powder form (for example in crystalline form, amorphous form, in the form of adsorbates, extrudates, granules and/or agglomerates) are charged in this case in a suitable apparatus, preferably in a fluidized bed or a mixer. The hydroformates are charged, optionally together with additives and other constituents. Plowshares, vanes, knife heads, kneading elements, runners, screws or the like ensure more or less intensive product mixture. Classic examples are intensive mixers, plowshare mixers, conical screw mixers or similar apparatuses.

Very flat, box- or trough-shaped constructions having one or more screws can also be used. Other construction types are rapid mixers, for example the Turbolizer® Mixer/Coater from Hosakawa Micron B.V., kneaders, edge runners and also all types of drum coaters or dragee drums.

Alternatively it is possible to mix the product via a movement of the entire vessel in what are termed free-fall mixers. Examples of these are coating disks, tumbling mixers, V mixers, drum mixers or the like. A further possibility is the use of pneumatic mixers. The mixing of solids is described, for example, in Ullmann's Encyclopedia of Industrial Chemistry, Sixth Edition, 2000, Mixing of Solids.

The coating can be carried out either directly in the apparatus downstream.

Said process can be carried out either continuously or batchwise (in corresponding batchwise or continuous mixers).

In individual cases it can be necessary, when applying the coating material or immediately thereafter/therebefore, to add powder-coating material such as talcum, silicates or similar to avoid sticking.

The coating material is metered/added, optionally together with other constituents, usually via devices for dropwise addition or spraying. Examples of these are lances, sprayheads, single-fluid or multiple-fluid nozzles, in rare cases rotating dropwise-addition or atomization devices. In the simplest case local addition as a concentrated jet is also possible.

The present invention relates to a process for preparing coated preparations comprising hydroformates in which coating materials, optionally with addition of further constituents, are charged in a suitable apparatus and hydroformates are added, optionally together with further constituents and/or additives.

In an embodiment of this process, the initially solid coating material is added to a suitable apparatus and melted or softened as a result of heating the wall of the apparatus, or the shaft, or as a result of the mechanical energy input. The hydroformates and, optionally, further constituents and/or additives are added and coated with the molten or softened coating material.

In an embodiment of this process, in addition to the coating material, supports are charged in the mixer and optionally premixed and as a result of high mechanical energy input in the same or separate apparatuses (examples are all previously mentioned mixers, but also slow-running mills and dryers), the hydroformates and also optionally further constituents and/or additives are coated.

The coating materials can be added at overpressure, atmospheric pressure or at reduced pressure, preferably at atmospheric pressure and reduced pressure.

In individual cases it can be advantageous to preheat or cool the hydroformates and also optionally further constituents and/or additives and/or the coating material (change of viscosity, change of wetting properties, influencing the solidification properties) and also to supply or remove heat via the vessel wall and/or the mixing tools. In individual cases it can be necessary to remove water vapor or solvent vapor. The wetting properties can also be changed by adding surface-active substances such as emulsifiers or the like.

To improve the coating properties it can be advantageous to evacuate the mixer and also optionally to blanket it with protective gas. Depending on the coating material this is to be repeated several times.

The hydroformates, optionally further constituents and/or additives and also the coating materials can be added as required at different sites in the apparatus.

The site of addition for coating materials or additives is varied in accordance with the requirements and is selected by a person skilled in the art. The above-described devices for trickling or spraying are disposed, according to requirements, above the product bed (top-spray process) or are disposed immersed in the product bed (from the side via apparatus walls, through channels in the mixing tools or from beneath via apparatus bottoms or gas-distribution plates).

The present invention relates to a process for preparing coated preparations comprising hydroformates in which the hydroformates (together with further constituents and/or additives) and the coating material are mixed in a suitable apparatus then extruded in an extrusion process and thereafter converted into granules by crushing and/or rounding.

In a further embodiment, after the mixing a pressing agglomeration with optional downstream comminution and fractionation is used for the granulation (for example tableting or roller compacting).

In a further embodiment of the present invention the preparations coated according the invention are prepared batchwise or continuously in fluidized beds. The motion of the particles arises from the possibly hot or cooled fluidizing gas. Suitable fluidizing gases are, for example, air or else inert gas (for example nitrogen). In individual cases it can be expedient to supply or remove heat via the vessel wall and also via heat-exchange surfaces immersed in the fluidized bed. Suitable fluidized beds and the peripherals required are part of the prior art.

The batchwise or continuous metering and optionally the preheating of the hydroformates, optionally of the other constituents and additives, is performed by above-described devices which are known to a person skilled in the art.

For example, the hydroformates can be charged in a fluidized bed. These are vortexed and coated by spraying on an aqueous or nonaqueous solution or dispersion or a melt of a suitable coating material.

Internals known from the prior art which reinforce a specific mixing of the solid to be coated are helpful. Examples of these are rotating displacement bodies, Wurster tubes or else specially fabricated gas-distribution plate geometries (inclination and/or perforation of the plate) or the reinforcement of the targeted solids motion by expediently arranged nozzles, for example tangentially arranged single-fluid or two-fluid or multiple-fluid nozzles.

Coated preparations comprising hydroformates can in individual cases advantageously be prepared in a combination of mixer and fluidized bed.

The present invention relates to a process for preparing coated preparations comprising hydroformates in which hydroformates, optionally together with further constituents and/or additives, are dispersed in melts of suitable coating materials and then the resultant dispersions finely divided and solidified.

In an embodiment of this process the hydroformates, optionally together with further constituents and/or additives, can be used in the form of a melt.

In a further embodiment the inventive coated preparations are obtained by suspending the hydroformates (and optionally the further constituents and/or additives) in melts of suitable coating materials and then atomizing and/or finely dividing and solidifying the resultant dispersions. Suitable coating materials in the form of melts are substances whose melting point is below the melting point of the hydroformates to be suspended. Examples are fats, waxes, oils, lipids, lipid-like and lipid-soluble substances having appropriate melting points.

These suspensions are then atomized in a cold gas stream, with and without using powder-coating materials, so that coated preparations comprising hydroformates are formed. These processes are known to a person skilled in the art, for example, under the terms spray cooling, spray solidification, prilling or melt encapsulation and also solidification on cooling belts, cooling rolls, tableting disks and tableting belts.

Preferably, the melts are prepared in a first step before the hydroformates are added and suspended. They can be suspended batchwise in a stirred tank or else continuously in, for example, pumps suitable therefor or simply in injectors and piping as a result of sufficiently high turbulence. It is also possible to use static mixers. The protective heating of the necessary plant components, including the lines and atomizing elements, is known to a person skilled in the art.

As cooling gas, use is preferably made of air and nitrogen. The gas can be conducted in cocurrent flow, countercurrent flow or cross flow. The process can be carried out in classic spraying towers, prilling towers or other vessels. Fluidized beds with and without hold-up are likewise suitable. The process can be operated batchwise or continuously. It is possible to separate off the solids in, for example, cyclones or filters. Alternatively, collecting the solids with and without aftercooling in fluidized beds or mixers is conceivable.

Suitable atomization elements are nozzles (single- and two-fluid nozzles or special construction types) and also atomizing wheels or atomizing disks or atomizing plates or atomizing baskets, or special construction types thereof.

In a further embodiment, the resultant dispersions are atomized and solidified in liquids in which neither the hydroformates nor the coating materials are soluble. A classic solid-liquid separation with subsequent drying leads to the inventive preparation.

The present invention further relates to a process for preparing coated preparations comprising hydroformates in which hydroformates, optionally further constituents and/or additives, are dispersed in a coating material, in particular a lipophilic coating material, emulsified in an aqueous solution of a protective colloid, preferably gelatin or/and gelatin derivatives or/and gelatin substitutes with addition of one or more substances selected from the group consisting of mono-, di- or polysaccharides, and then subjected to a spray drying.

In this process, preferably, very fine-grained hydroformates are used which are obtained, for example, by precipitation, crystallization, spray drying or grinding.

In one embodiment, before the dispersion in the lipophilic component, one or more emulsifiers and/or stabilizers can be added to the hydroformates.

Suitable lipophilic coating materials are melts of fats, oils, waxes, lipids, lipid-like and lipid-soluble substances having a melting point below the melting point of the hydroformates used.

The resultant dispersions (the hydroformate-containing oil droplets) are, in a subsequent process step, emulsified in an aqueous solution of a protective colloid, preferably gelatin or/and gelatin derivatives or/and gelatin substitutes, with addition of one or more substances selected from the group consisting of mono-, di- or polysaccharides, preferably corn starch. The resultant emulsions are subjected to shaping by spraying and subsequent or simultaneous drying.

In a further embodiment the inventively coated preparations comprise the hydroformates bound to a support.

The support-bound preparations are prepared by preparation processes known to a person skilled in the art, for example, by adsorbing the inventive preparations in liquid form to the support substances.

Use

The inventive coated or liquid preparations are suitable for use in feeds for animals (animal feeds). Examples are: pigs, ruminants, poultry and other domestic and farm animals. The inventive preparations are suitable in particular for fattening poultry (broilers, turkeys, geese), and also for fattening pigs.

The inventive coated or liquid preparations are suitable, in particular, as additive to animal feeds in the form of feed additives.

Feed additives are, under the German Feedstuff Act, in particular those substances which are intended to be added to feeds individually or in the form of preparations for
affecting the properties of the feeds or of the animal products,
covering the requirement of the animals for certain nutrients or active compounds or improving animal production, in particular by action on the gastrointestinal flora or the digestibility of the feeds or by reducing nuisance due to excretions of the animals, or
achieving particular nutritional purposes or covering defined temporary nutritional requirements of the animals.

Feed additives are also considered to be substances which are permitted as additives by regulation under § 4 paragraph 1 No. 3 b of the German Feedstuff Act.

The inventive coated or liquid preparations are suitable in particular as additive to premixes for animal feeds. Premixes are mixtures of minerals, vitamins, amino acids, trace elements and also optionally enzymes. Using the inventive preparations it is possible to prepare premixes comprising hydroformates.

The present invention further relates to a process for preparing a hydroformate-containing feed and/or feed additive, which comprises

(i) adding a coated preparation comprising hydroformates to a premix

(ii) mixing the resultant premix with the remaining constituents of the feed and/or feed additive.

The present invention further relates to a process for preparing a hydroformate-containing feed and/or feed additive, which comprises

(i) adding a liquid preparation comprising hydroformates to a premix

(ii) mixing the resultant premix with the remaining constituents of the feed and/or feed additive.

The present invention further relates to a process for preparing a hydroformate-containing feed and/or feed additive, which comprises mixing a liquid preparation comprising hydrofonmates with the remaining constituents of the feed and/or feed additive.

In a preferred embodiment, the liquid preparations can be mixed directly with the feed or added to the animals' drinking water.

The inventive coated or liquid preparations are suitable, in particular, as what are termed “acidifiers”. Acidifiers are those substances which lower the pH. These include not only those substances which lower the pH in the substrate (for example animal feed) but also those which lower the pH in the gastrointestinal tract of the animal.

The inventive coated or liquid preparations are suitable, in particular, as performance enhancers. In a preferred embodiment the inventive preparations are used as performance enhancers for pigs and poultry.

The inventive coated or liquid preparations are suitable, in particular, as feed additives which lower the pH in the gastrointestinal tract of the animal and as a result contribute to stabilizing an advantageous intestinal microflora. In addition, owing to the dissociation capacity of the inventive coated or liquid preparations, an antimicrobial effect can result, in particular on unwanted bacteria (for example, E. coli). In a preferred embodiment the inventive coated or liquid preparations are used so as to reduce the pH beyond the stomach, still in the region of the further digestive tract. This is in particular advantageous in the case of poultry.

Animal feeds are composed in such a manner that the corresponding requirement for nutrients is optimally covered for the respective type of animal. Generally, plant feed components such as corn meal, wheat meal or barley meal, whole soybean meal, soybean extraction meal, linseed extraction meal, rapeseed extraction meal, grass meal or pea meal are selected as crude protein sources. To ensure an appropriate energy content of the feedstuff, soybean oil or other animal or vegetable fats are added. Since the plant protein sources contain some essential amino acids only in an inadequate amount, feeds are frequently enriched with amino acids. These are especially lysine and methionine. To ensure the mineral and vitamin supply to the farm animals, minerals and vitamins are added in addition. The type and amount of added minerals and vitamins depends on the animal species and is known to those skilled in the art (see, for example, Jeroch et al., Emahrung landwirtschaftlicher Nutztiere [Nutrition of agricultural farmed animals], Ulmer, UTB). To cover the nutrient and energy requirements, complete feeds can be used which contain all nutrients in a ratio to one another for covering requirements. It can form the sole feed of the animals. Alternatively, it can be added as a supplemental feed to a cereal grain feed. These are protein-, mineral- and vitamin-rich feed mixes which expediently supplement the feed.

The inventive coated or liquid preparations are also suitable as preservatives, in particular as preservatives for green fodder and/or animal feed.

It has been found that the inventive coated or liquid preparations can advantageously be used in the production of silage. They accelerate the lactic fermentation and prevent secondary fermentation. The invention therefore further relates to the use of the inventive coated or liquid preparations as silage additive.

The present invention further relates to the use of the inventive coated or liquid preparations in fertilizers.

### EXAMPLE 1

### Preparing Coated Preparations

Melt agglomeration in the R02 intensive mixer from Eirich

1000 kg of trisodium hydrogenformate are charged in the mixer and, via the electrical trace heating, heated to a product temperature of 50° C. with rotating mixer pot (n=84 min⁻¹). 0.290 kg of Vegeol PR 273 were added as binder/coating material, after preheating to T=85° C., molten, in a full jet at a spray rate of 40 g/min. During the melt addition, mixer pot (n=84 min⁻¹) and star vortexer (n=900 min⁻¹) contrarotated. After completion of the melt addition, the product was regranulated over 3 min with mixer pot (n=84 min⁻¹) and star vortexer (n=900 min⁻¹) rotating cocurrently. A product temperature of T=58° C. was established. The cooling to 35° C. was performed with the mixer stationary. Thereafter stable agglomerates made up of primary agglomerates were present screened.

Bulk density: 610 kg/m³

Sieve analysis flat oscillating screen:

Mass fraction of fines (<0.5 mm): 12.5%.

Mass fraction of coarse material (>1.4 mm): 24.5%

Mass fraction of useful material: (0.5-1.4 mm): 63%

### EXAMPLE 2

### Feeding Experiment Using Coated Preparations

A feeding experiment was carried out using one-day-old chickens. Trisodium hydrogenformate was coated with 0, 15 and 30% by weight hydrogenated soybean oil. Per dose, 2 repetitions were carried out, and 10 chickens were used in each feeding experiment.

A standard wheat-soy diet was used as negative control. The coated trisodium hydrogenformate preparations were added to the diet in exchange for cellulose (1% in the negative control).

The improvements in weight gain and feed utilization after 21 days of the feeding experiment are shown in the table below:

Feeding results compared with the negative control [%] after 21 days.

| Coating [% by weight] | | |
|---|---|---|
| 0 | 15 | 30 |

| | | | | | | |
|---|---|---|---|---|---|---|
| Trisodium | 0.4 | 0.8 | 0.4 | 0.8 | 0.4 | 0.8 |
| hydrogenformate [% by | | | | | | |
| weight] | | | | | | |
| Weight gain | +2 | +2 | +4 | +4 | +6 | +4 |
| FCR | +2 | +5 | +5 | +1 | +4 | +3 |
| (feed conversion rate) | | | | | | |

### EXAMPLE 3

### Liquid Preparation

40 g of trisodium hydrogenformate are mixed with 60 g of water. This produces a solution comprising

40% by weight of trisodium hydrogenformate

60% by weight of water.

### EXAMPLE 4

### Liquid Preparation Comprising Benzoic Acid

5 g of benzoic acid are added to the solution of example 3. This corresponds to a content of 5% by weight based on the entire solution.

1. A coated preparation comprising
at least one hydroformate of the general formula (I)
M₃[HCOO]₃.HCOOH (I)
where M=Na, K, Cs, NH₄.

2. A preparation as claimed in claim 1 wherein the hydroformate is trisodium hydroformate.

3. A preparation as claimed in claim 1 wherein the preparation comprises further constituents and/or additives and/or supports.

4. A preparation as claimed in claim 1, wherein the coating material is at least one compound which is selected from the group consisting of
a) polyalkylene glycols;
b) poly(alkylene oxide) polymers or copolymers having a number-average molecular weight of from about 4000 to 20 000;
c) substituted polystyrenes, maleic acid derivatives and also styrene-maleic acid copolymers;
d) polyvinylpyrrolidones having a number-average molecular weight of from about 7000 to 1 000 000;
e) vinylpyrrolidone/vinyl acetate copolymers having a number-average molecular weight of from about 30 000 to 100 000;
f) poly(vinyl alcohol) having a number-average molecular weight of from about 10 000 to 200 000, poly(vinyl phthalate)s;
g) hyroxypropylmethylcellulose having a number-average molecular weight of from about 6000 to 80 000;
h) alkyl (meth)acrylate polymers and copolymers having a number-average molecular weight of from about 100 000 to 1 000 000
i) poly(vinyl acetate) having a number-average molecular weight of from about 250 000 to 700 000, optionally stabilized with polyvinylpyrrolidone;
j) polyalkylenes;
k) phenoxyacetic acid-formaldehyde resin;
l) cellulose derivatives, such as ethylcellulose, ethylmethylcellulose, methylcellulose, hydroxypropylcellulose, hydroxypropylmethylcellulose, carboxymethylcellulose, cellulose acetate phthalate;
m) animal, vegetable or synthetic fats;
n) animal, plant or synthetic waxes or chemically modified animal, plant waxes such as beeswax, candelilla wax, carnauba wax, montan ester wax and rice germ oil wax, spermaceti, lanolin, jojoba wax, sasol wax, Japan wax or Japan wax substitute;
o) animal or plant proteins;
p) mono- and disaccharides, oligosaccharides, polysaccharides;
q) vegetable oils;
r) synthetic or semisynthetic oils;
s) animal oils;
t) hardened (hydrogenated or partially hydrogenated) oils/fats;
u) lacquer coatings;
v) fatty acids, not only saturated but also monounsaturated and polyunsaturated C₆- to C₂₄-carboxylic acids;
w) silicic acids;
x) benzoic acid and/or salts of benzoic acid and/or esters of benzoic acid and/or derivatives of benzoic acid and/or salts of benzoic acid derivatives and/or esters of benzoic acid derivatives.

5. A preparation as claimed in claim 1, which is a powder with a mean particle size of from 1 μm to 10,000 μm.

6. A process for preparing coated preparations as claimed in claim 1, which comprises
(i) charging at least one hydroformate, optionally with admixture of further constituents and/or additives
(ii) coating the resultant mixture with a coating material, optionally together with further constituents.

7. A process for preparing coated preparations as claimed in claim 1, which comprises
(i) charging coating material, optionally with addition of further constituents, in a suitable apparatus
(ii) adding at least one hydroformate, optionally together with further constituents and/or additives.

8. A process for preparing preparations as claimed in claim 1, which comprises applying the hydroformates, before the coating, to a support material.

9. A process for preparing coated preparations as claimed in claim 1, which comprises
(i) dispersing at least one hydroformate, optionally together with further constituents and/or additives, in melts of suitable coating materials
(ii) finely dividing and solidifying the resultant dispersions.

10. A process for preparing coated preparations as claimed in claim 1, which comprises
(i) dispersing at least one hydroformate, optionally together with further constituents and/or additives, in a coating material,
(ii) emulsifying it in an aqueous solution of a protective colloid
(iii) and subjecting it to shaping by spraying and subsequent or simultaneous drying.

11. A process for preparing coated preparations as claimed in claim 1, which comprises coating at least one hydroformate by desublimating the coating material.

12. (canceled)

13. (canceled)

14. A process for preparing a feed and/or feed additive comprising at least one hydroformate, which comprises
(i) adding a preparation as claimed in claim 1 to a premix
(ii) mixing the resultant premix with the remaining constituents of the feed and/or feed additive.

15. An animal feed comprising a preparation as claimed in claim 1.

16.-20. (canceled)

21. The animal feed as claimed in claim 15, wherein the feed can be feed to pigs, poultry or calves.

22. A preparation as claimed claim 1, wherein the coating material is at least one compound which is selected from the group consisting of
a) polyalkylene glycolshaving a number-average molecular weight of from about 400 to 15 000;
b) block copolymers of polyoxyethylene and polyoxypropylene having a number-average molecular weight of from about 4000 to 20 000;
c) substituted polystyrenes, maleic acid derivatives and also styrene-maleic acid copolymers;
d) polyvinylpyrrolidones having a number-average molecular weight of from about 7000 to 1 000 000;
e) vinylpyrrolidone/vinyl acetate copolymers having a number-average molecular weight of from about 30 000 to 100 000;
f) poly(vinyl alcohol) having a number-average molecular weight of from about 10 000 to 200 000, poly(vinyl phthalate)s;
g) hyroxypropylmethylcellulose having a number-average molecular weight of from about 6000 to 80 000;
h) alkyl (meth)acrylate polymers and copolymers having a number-average molecular weight of from about 100 000 to 1 000 000, in particular ethyl acrylate/methyl methacrylate copolymers and methacrylate/ethyl acrylate copolymers;
i) poly(vinyl acetate) having a number-average molecular weight of from about 250 000 to 700 000, optionally stabilized with polyvinylpyrrolidone;
j) polyethylenes;
k) phenoxyacetic acid-formaldehyde resin;
l) cellulose derivatives, such as ethylcellulose, ethylmethylcellulose, methylcellulose, hydroxypropylcellulose, hydroxypropylmethylcellulose, carboxymethylcellulose, cellulose acetate phthalate;
m) animal, vegetable or synthetic fats;
n) animal, plant or synthetic waxes or chemically modified animal, plant waxes such as beeswax, candelilla wax, carnauba wax, montan ester wax and rice germ oil wax, spermaceti, lanolin, jojoba wax, sasol wax, Japan wax or Japan wax substitute;
o) gelatin, gelatin derivatives, gelatin substitutes, casein, whey, keratin, soybean protein; zein or wheat protein;
p) starches, modified starches, pectins, alginates, chitosan, or carrageenans;
q) sunflower oil, thistle oil, cottonseed oil, soybean oil, corn germ oil, olive oil, rape(seed) oil, linseed oil, coconut oil, palm kernel oil, or palm oil;
r) medium-chain triglycerides or mineral oils;
s) herring oil, sardine oil or whale oil;
t) hydrogenated palm oil, hydrogenated cottonseed oil, or hydrogenated soybean oil;
u) terpenes;
v) fatty acids, not only saturated but also monounsaturated and polyunsaturated C₆- to C₂₄-carboxylic acids;
w) silicic acids;
x) benzoic acid and/or salts of benzoic acid and/or esters of benzoic acid and/or derivatives of benzoic acid and/or salts of benzoic acid derivatives and/or esters of benzoic acid derivatives.

23. A performance enhancer and/or growth promoter which comprises the preparation as claimed in claim 1.

24. An acidifier which comprises the preparation as claimed in claim 1.

25. A preservative which comprises the preparation as claimed in claim 1.

26. A silage additive which comprises the preparation as claimed in claim 1.

27. A fertilizer which comprises the preparation as claimed in claim 1.

28. A preparation as claimed in claim 2, which is a powder with a mean particle size of from 20 μm to 5,000 μm and coating material is at least one compound which is selected from the group consisting of
a) polyethylene glycols, having a number-average molecular weight of from about 400 to 10 000;
b) block copolymers of polyoxyethylene and polyoxypropylene having a number-average molecular weight of from about 4000 to 20 000;
c) substituted polystyrenes, maleic acid derivatives and also styrene-maleic acid copolymers;
d) polyvinylpyrrolidones having a number-average molecular weight of from about 7000 to 1 000 000;
e) vinylpyrrolidone/vinyl acetate copolymers having a number-average molecular weight of from about 30 000 to 100 000;
f) poly(vinyl alcohol) having a number-average molecular weight of from about 10 000 to 200 000, poly(vinyl phthalate)s;
g) hyroxypropylmethylcellulose having a number-average molecular weight of from about 6000 to 80 000;
h) alkyl (meth)acrylate polymers and copolymers having a number-average molecular weight of from about 100 000 to 1 000 000, in particular ethyl acrylate/methyl methacrylate copolymers and methacrylate/ethyl acrylate copolymers;
i) poly(vinyl acetate) having a number-average molecular weight of from about 250 000 to 700 000, optionally stabilized with polyvinylpyrrolidone;
j) polyethylenes;
k) phenoxyacetic acid-formaldehyde resin;
l) cellulose derivatives, such as ethylcellulose, ethylmethylcellulose, methylcellulose, hydroxypropylcellulose, hydroxypropylmethylcellulose, carboxymethylcellulose, cellulose acetate phthalate;
m) animal, vegetable or synthetic fats;
n) animal, plant or synthetic waxes or chemically modified animal, plant waxes such as beeswax, candelilla wax, carnauba wax, montan ester wax and rice germ oil wax, spermaceti, lanolin, jojoba wax, sasol wax, Japan wax or Japan wax substitute;
o) gelatin, gelatin derivatives, gelatin substitutes, casein, whey, keratin, soybean protein; zein or wheat protein;
p) starches, modified starches, pectins, alginates, chitosan, or carrageenans;
q) sunflower oil, thistle oil, cottonseed oil, soybean oil, corn germ oil, olive oil, rape(seed) oil, linseed oil, coconut oil, palm kernel oil, or palm oil;
r) medium-chain triglycerides or mineral oils;
s) herring oil, sardine oil or whale oil;
t) hydrogenated palm oil, hydrogenated cottonseed oil, or hydrogenated soybean oil;
u) shellac, Tolu balsam, Peru balsam, sandarac or silicone resins;
v) fatty acids, not only saturated but also monounsaturated and polyunsaturated C₆- to C₂₄-carboxylic acids;
w) silicic acids;
x) benzoic acid and/or salts of benzoic acid and/or esters of benzoic acid and/or derivatives of benzoic acid and/or salts of benzoic acid derivatives and/or esters of benzoic acid derivatives.

29. A process for preparing coated preparations as claimed in claim 2, which comprises
(i) dispersing at least one hydroformate, optionally together with further constituents and/or additives, in a lipophilic coating material,
(ii) emulsifying it in an aqueous solution of gelatin or/and gelatin derivatives or/and gelatin substitutes with addition of one or more substances selected from the group consisting of mono-, di- or polysaccharides
(iii) and subjecting it to shaping by spraying and subsequent or simultaneous drying.
